(19) United States
(12) Patent Application Publication
(10) Pub. No.: US 2004/0267108 A1
(43) Pub. Date: Dec. 30, 2004

(54) Non-invasive electro-mechanical tonometer for measurement of intraocular pressure

(76) Inventor: Eugene John Moore, Old Bridge, NJ (US)

Correspondence Address: McCARTER & ENGLISH, LLP, Attn.: Anita Lomartra, CityPlace I, 185 Asylum Street, Hartford, CT, 06103, US

(21) Appl. No.: 10/803,681

(22) Filed: Mar. 18, 2004

Related U.S. Application Data
(60) Provisional application No. 60/456,460, filed on Mar. 21, 2003.

(51) Int. Cl. A61B 3/16

(52) U.S. Cl. 600/398

(57) ABSTRACT

A tonometer for measuring the intraocular pressure (IOP) of an eye through the eyelid of an individual, i.e., in a non-invasive manner, includes a frame, a strain gage mounted with respect to the frame for measuring a force, a linear variable displacement transducer mounted with respect to the frame, and a processing unit in communication with the strain gage and the linear variable displacement transducer. The linear variable displacement transducer communicates with an axially movable sensing tip for measuring a distance. The processing unit operates to (i) time-synchronize signals received from the strain gage and the linear variable displacement transducer, and (ii) identify a change in the relationship between time-synchronized measurements of the force and distance. The change in the force/distance relationship correlates with the intraocular pressure of a patient and may be observed as an inflection or trough-like area in a force-distance graph.

### CROSS-REFERENCE TO RELATED APPLICATION

The present application claims the benefit of a co-pending provisional patent application entitled “Non-Invasive Electro-Mechanical Tonometer for Measuring Intraocular Pressure,” which was filed on Mar. 21, 2003 and assigned Ser. No. 60/456,460. The contents of the foregoing provisional patent application are incorporated herein by reference.

### BACKGROUND

1. Technical Field

The present disclosure is directed to a device for measuring intraocular pressure and, more particularly, to a tonometer for measuring the intraocular pressure of an eye through the eyelid of an individual.

2. Discussion of Background Art

Glaucoma is a disease of the optic nerve in which the nerve cells in the front of the optic nerve (the ganglion cells) die. The process is currently irreversible and open angle glaucoma is a leading cause of blindness in the United States. Statistical studies show that at least three million people in the United States suffer from open angle glaucoma, while as many as sixty million people worldwide are afflicted with open angle glaucoma. Normal intraocular pressure (“IOP”) is generally in the range of 10 to 20 mm Hg. Increased intraocular pressure is present in most cases of glaucoma. While measurements above the 10-20mm Hg range may reflect and/or predict glaucoma, only about 10% of people with IOP levels between 21 and 30 mm Hg will actually develop glaucoma. At a minimum, these higher readings are useful in identifying individuals at considerable risk for glaucoma.

Open-angle glaucoma generally involves a series of blockages in the tiny drainage channels in the trabecular meshwork, which is a sponge-like, porous network responsible for most of the outflow of ocular fluid from the eye. Due to the blockages, an imbalance occurs as aqueous humor continues to be produced but does not drain out efficiently. In some cases, an imbalance may occur because the eye produces too much aqueous humor. In either case, fluid in the eye's anterior chamber builds up and increases pressure within the eye which, in turn, exerts force on the optic nerve at the back of the eye. Over time, the persistent pressure and/or other factors irreversibly damage the delicate long fibers of the optic nerve, called axons, which convey images to the brain. As these axons die, the small cup-like head of the optic nerve may eventually collapse into an enlarged irregular shape. If untreated, eventually the nerve deteriorates until a person loses sight, first in the peripheral vision and, if the condition becomes severe, central vision. Blindness is generally preventable with early treatment of the conditions described herein.

Generally, existing methods and devices for measuring a patient's IOP operate by measuring the force required to generate a defined deformation of the cornea, and calculating the IOP based on such force measurement. Devices of the foregoing type have been disclosed by Goldmann, Schiotz, Perkins, Mackay-Marg and Draeger. Generally, the eye is anesthetized before undertaking the foregoing force measurements.

In the field, it is generally recognized that intraocular pressure is most accurately measured by Goldmann applanation tonometry. The Goldmann tonometer is a biprism mounted on a standard slit-lamp, which is used to applanate (flatten) the anaesthetized fluorescein stained cornea. The IOP calculation is generally based on the Imbert-Fick principle, whereby an external force (exerted by the tonometer) against a sphere (the eye) equals the pressure within the sphere times the area flattened by the force (3.06 sq. mm of the cornea). In practice, a clinician anesthetizes a patient's eye and moves the tonometer's tip toward the subject's cornea until the applantor tip is viewed to have applanated a precise area of the cornea, at which point a pressure reading is taken/calculated.

An alternative pressure measurement method involves indentation tonometry using a Schiotz tonometer. A preset weight is placed on the tonometer which is placed on the anaesthetized cornea. The amount that the plunger sinks into the eye is measured and the reading is converted to mm Hg using a conversion table. The further the weight sinks into the eye, the “softer” the eye, thereby reflecting a lower IOP. The Schiotz tonometry method is frequently used in emergency departments where applanation tonometry is not available.

Individuals generally undergo periodic eye examinations that include IOP measurements by trained clinicians. For individuals that have been diagnosed with glaucoma and/or have exhibited a proclivity for high IOP readings, IOP measurements may be taken on a pre-scheduled basis, e.g., every three months. Based on a patient's IOP reading, the clinician may prescribe medicated drops to address an elevated IOP condition. However, diurnal variations in IOP are known and may impact on the reliability of a clinician's periodic IOP measurements. For example, it has been shown that IOP readings are highest in the early morning, e.g., just after waking, but decline within an hour. Also, studies have shown that substantial IOP fluctuations may occur outside the clinical/office setting. Thus, the reliability and effectiveness of periodic, e.g., quarterly, office-based IOP measurements may pose issues for diagnosis and/or treatment of glaucoma and other pressure-based optical conditions.

Various devices for measuring IOP have been disclosed in the patent literature. For example, U.S. Pat. No. 6,093,147 to Kontiola discloses a probe that may be utilized outside of an office/clinical setting to measure IOP. The Kontiola probe is set in motion from a distance of a few millimeters to impact the eye or eyelid, which results in a change in the probe's motion. The Kontiola '147 device measures the continuous motion of the probe from which the intraocular pressure is then derived using known physical laws. Thus, according to the Kontiola '147 patent, an overall analysis is made of the motion during and after impact, including the effects of gravitation and friction on the probe.

U.S. Pat. No. 5,735,275 to Ballou et al. also discloses a tonometer for measuring intraocular pressure of an eye is described. The Ballou '275 tonometer includes an eyepiece having a conduit extending through an outer surface which is concave-shaped to conform to the geometry of an eyeball. The tonometer includes a plunger extending through the conduit that features a head at one end and a probe at the opposite end, the probe extending beyond the outer surface of the eyepiece so as to contact the eyeball when the eyepiece is positioned to conform to the eyeball. The tonometer also includes a fluid reservoir that is sealed by a membrane in juxtaposition with the head of the plunger, and an indicator positioned in the fluid reservoir for measuring a level of fluid that is proportional to an intraocular pressure of the eyeball when the eyepiece is positioned to conform to the eyeball. According to the Ballou '275 patent, the distance by which the plunger is displaced is proportional to the force exerted on the plunger caused by contacting the eyeball to the eyepiece, which is in turn proportional to the intraocular pressure of the eyeball and is reflected by the indicator within the fluid reservoir.

U.S. Pat. No. 5,349,955 to Suzuki discloses a tonometer that includes a pressure rod that is urged against a closed eye at a constant velocity by a spring. When a trigger is pulled by an operator, a slide bed is moved forward by the force of the spring, whereby a pressure rod presses against the eyelid, thereby exerting pressure on the eyeball under a load that is detected by a load sensor. The signals output by the load sensor are subjected to A/D conversion by an A/D converter and are then input to a signal processing circuit. The change in load relative to time is obtained and the correlation between differential of load with time and intraocular pressure is used to convert the to an intraocular pressure value. An eye fixation lamp is used to fix the vision of the other eye to thereby ensure that the cornea of the eye being examined faces to the front, although the eye is closed, thereby helping to improve measurement accuracy.

Despite efforts to date, a need remains for a tonometry device that provides reliable IOP readings in a non-clinical setting. In addition, a need remains for a tonometry device that provides reliable IOP readings in a non-invasive manner.

These and other needs are satisfied by the tonometry devices disclosed herein and described hereinafter.

### SUMMARY OF THE DISCLOSURE

According to the present disclosure, an advantageous tonometer for measuring the intraocular pressure (IOP) of an eye through the eyelid of an individual, i.e., in a non-invasive manner, is disclosed. The tonometer generally includes, but is not limited to, a frame, a strain gage mounted with respect to the frame for measuring a force, a linear variable displacement transducer mounted with respect to the frame, and a processing unit in communication with the strain gage and the linear variable displacement transducer. The linear variable displacement transducer communicates with an axially movable sensing tip for measuring a distance. The processing unit is advantageously programmed to (i) time-synchronize signals received from the strain gage and the linear variable displacement transducer, and (ii) identify a change in the relationship between time-synchronized measurements of the force and distance. This change in the force/distance relationship, which correlates with the intraocular pressure of a patient, may be observed in any of a variety of ways, such as, for example, an observed inflection or trough-like area in a force-distance graph.

In exemplary embodiments of the present disclosure, the tonometer includes a strain gage stop and/or a linear variable displacement transducer (LVDT) stop. The stop(s) function to maintain operation of the strain gage and/or the LVDT within their respective operating ranges. The strain gage stop may advantageously include an adjustment mechanism, e.g., a fine adjustment screw, to facilitate variations in strain gage stop positioning. The strain gage and LVDT communicate with the processing unit by way of data transmission wires. The processing unit typically provides signal processing functionalities, including signal amplification, low pass signal filtering, signal rectification and signal processing through a digital acquisition card. The digital acquisition card feeds the processed signals to software that operates to time-synchronize the signals received from the strain gage and the LVDT, and to identify a change or an inflection in the relationship between time-synchronized force and distance measurements. The software for identifying a change in the force/distance relationship may include spreadsheet functionality. According to the present disclosure, the change in the force/distance relationship correlates with the intraocular pressure of a patient.

Additional structural and functional features and attributes of the disclosed tonometers will be apparent from the detailed description which follows, particularly when read in light of the figures appended hereto.

### BRIEF DESCRIPTION OF THE FIGURES

So that those having ordinary skill in the art to which the present disclosure pertains will more readily understand how to make and use the devices described herein, exemplary embodiments thereof will be described in detail with reference to the appended figures, wherein:

FIG. 1 is a schematic diagram of an exemplary tonometer according to the present disclosure;

FIG. 2 is a schematic diagram of exemplary a signal processing design for use with a tonometer according to the present disclosure;

FIG. 3 is a plot of calibration linearity for a linear variable displacement transducer (LVDT) for an exemplary embodiment of the present disclosure;

FIG. 4 is a plot of calibration linearity for a strain gauge for an exemplary embodiment of the present disclosure; and

FIG. 5 is a plot of pressure versus distance for an exemplary tonometer according to the present disclosure.

### DETAILED DESCRIPTION OF EXEMPLARY EMBODIMENT(S)

The present disclosure provides an advantageous device for measuring intraocular pressure (IOP). The disclosed device generally takes the form of a tonometer that is designed and configured for measuring the intraocular pressure of an eye through the eyelid of an individual, i.e., in a non-invasive and non-anesthetized manner. The tonometer of the present disclosure permits users to “self-measure” IOP, i.e., obtain IOP measurements without the assistance of a health care professional. Thus, the disclosed tonometer permits individuals to monitor variations in IOP to a degree that is generally impossible and/or impractical, thereby gaining IOP data that may be used to evaluate and assess the magnitude or significance of diurnal variations in IOP. In this way, an individual is better able to monitor and manage glaucoma or glaucoma-type conditions and/or ailments.

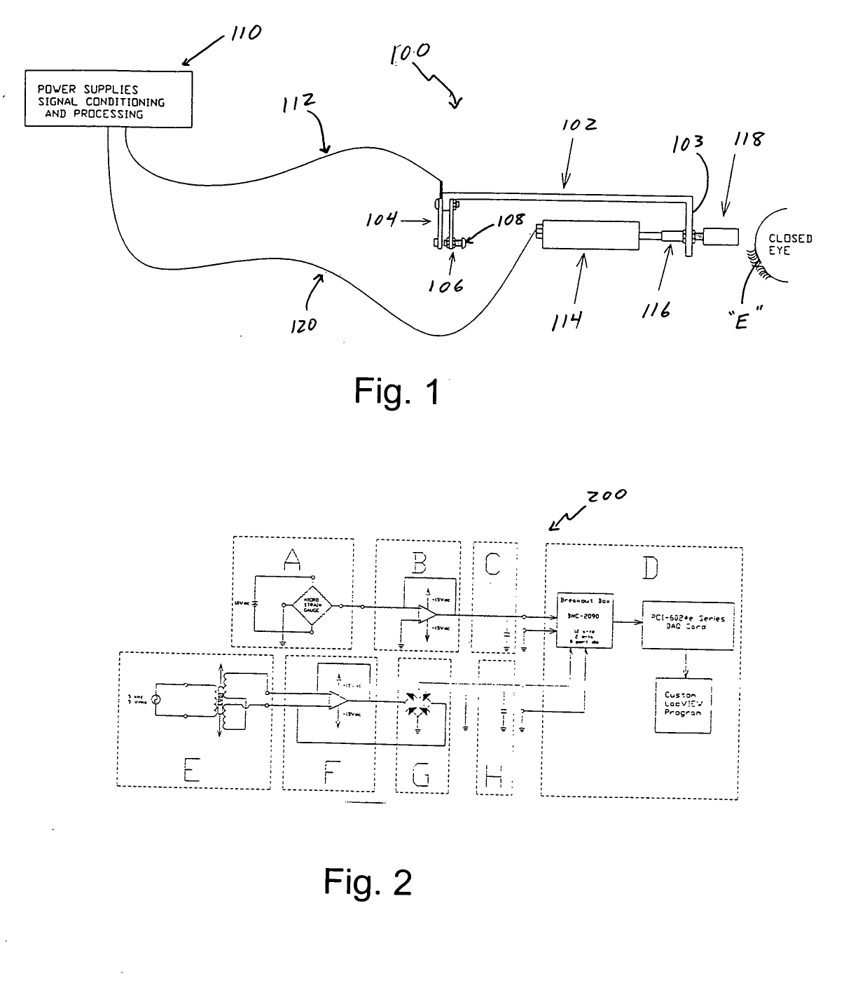

The disclosed tonometer generally includes a frame, a strain gage, a linear variable displacement transducer, an eyelid contact tip, and a processing unit/processor that operates to obtain and process relevant data. Although the disclosed tonometer may vary in structural arrangement and/or detail without departing from the spirit and/or scope of the present disclosure, reference is made to FIG. 1 wherein an exemplary tonometer 100 according to the present disclosure is schematically depicted.

Tonometer 100 includes frame 102 that defines a substantially L-shaped geometry and that functions, inter alia, to translate the force applied at the strain gage to the sensing tip, as described herein. Frame 102 may be fabricated from any suitable rigid material, e.g., a rigid aluminum. A strain gage 104 is mounted at a distal end of frame 102. A variety of mounting mechanisms may be used to secure strain gage 104 with respect to frame 102, e.g., a brass screw/nut combination or the like. In an exemplary embodiment of the present disclosure, strain gage 104 may take the form of a micro strain gage that is designed to measure small forces, e.g., in the 0 to 50 gram range. A suitable strain gage is commercially available from Strain Measurement Devices (Model No. 2239; Meriden, Conn.), which advantageously measures with a resolution to below 10 mg.

A strain gage stop 106 is typically mounted with respect to frame 104 in an orientation that is adjacent to strain gage 104. In the disclosed embodiment, strain gage 104 and strain gage stop 106 are mounted to frame 102 with the same screw/nut combination. Strain gage stop 106 includes an adjustment mechanism 108, e.g., a fine adjustment screw (e.g., brass) that functions to protect the mechanical range of strain gage 104. An exemplary strain gage stop 106 according to the present disclosure is fabricated from rigid aluminum. Strain gage 104 communicates with a processor/processing unit 110, e.g., over communication wire 112. In an alternative embodiment of the present disclosure, wireless communication may be implemented, thereby obviating the need for communication wire 112, as will be apparent to persons skilled in the art. The nature of the communication between processor 110 and strain gage 104 is discussed in greater detail below.

With further reference to FIG. 1, linear variable displacement transducer (LVDT) 114 is mounted with respect to downwardly extending arm 103 of frame 102. LVDT 114 cooperates with an LVDT stop 116 that may be positioned immediately adjacent arm 103. In an exemplary embodiment of the present disclosure, LVDT stop 116 may be precision cut from plastic tubing or electrical shrink tubing, and is typically used to initialize the linear or axial position of LVDT 114. As used herein, the LVDT 114 is substantially aligned with the longitudinal axis of tonometer 100. LVDT 114 is generally an inductive sensing device that produces an alternating current (AC) output voltage that is proportional to the mechanical displacement of a small iron core. In a typical LVDT for use according to the present disclosure, one primary and two secondary coils are symmetrically arranged to form a hollow cylinder. A magnetic nickel-iron core moves axially within the cylinder in response to mechanical displacement of a probe tip. LVDT measurements are based on inductance effects that do not involve flexing wires or sliding electrical contacts, thereby reducing the potential for undesirable “noise.” An exemplary LVDT for use according to the present disclosure is available from Cooper Instruments (Model D5/100K; Warrenton, Va.), which has a range of +/−0.1 inches and a resolution of less than 0.001 inches.

Thus, as shown in FIG. 1, tonometer 100 includes a sensing tip 118 that is adapted for axial displacement relative to LVDT 114. In an exemplary embodiment of the present disclosure, sensing tip 118 is machined from a polycarbonate material, e.g., Lexan® polycarbonate (General Electric Co., Pittsfield, Mass.). Sensing tip 118 generally includes a substantially flat circular end of a known area for applying force to the cornea of the eye, through the eyelid, in a non-invasive manner. LVDT 114 is advantageously designed to measure small precise changes in distance, e.g., on the order of 0.1 inches. LVDT 114 communicates with processor 110 by way of wire 120. Both wires 112, 120 are typically coated to enhance shielding properties thereof.

In use, measurements are simultaneously captured by strain gage 104 and LVDT 114, such measurements being communicated to processor 110 over wires 112, 120, respectively. The combined measurements of LVDT 114 and strain gage 104, together with a circuit embodied in processor 110 for signal conditioning and programming for processing such measurement data, e.g., a LabVIEW program, make it possible for both signals to be time synchronized, saved and analyzed. With reference to FIG. 1, sensing tip 118 is applied to a closed eyelid “E”, typically directly over the cornea. The size and/or geometry of sensing tip 118 is believed to play a large role in measurement accuracy and repeatability, because a larger tip is more susceptible to misalignment and, therefore, distributes force over a smaller and less certain area of the tip. These issues introduce potential inaccuracies as the measured force is converted to a pressure value. A smaller tip is likely to reduce the amount of error produced by misalignment and is also likely to translate into greater reproducibility.

The force is gently increased by the user until the necessary corneal deformation takes place. Force and distance data is communicated to processor 110 and the data is sampled at a rapid rate, e.g., 1000 samples per second, by the program embodied in processor 110. In an exemplary embodiment of the present disclosure, the stream of sampled data is input to a spreadsheet file, e.g., an Excel™ file (Microsoft Corporation; Redmond, Wash.).

According to an exemplary data processing methodology according to the present disclosure, the force measurement and distance measurements are synchronized. A conversion is performed to change the force voltages to grams using the calibration data, and then to pressure (mm Hg) using the following formula:

$$M ( g ) \times - \frac{0.001 ( k g \text{/} g )}{{Π ( 0.003 )}^{2} ( {m}^{2} )} \times 9.8 m \text{/} {s}^{2} \times \frac{1 ( mm Hg )}{133.3 ( kg * {m}^{- 1} {s}^{- 2} )} = M ( g ) \times 2.60 ( mm Hg \text{/} g )$$

With reference to FIG. 2, schematic diagram 200 depicts an exemplary electronic component layout for processing electrical signals, with respective signal conditioning and data interface components, according to the present disclosure. A 10 Volt direct current (DC) power supply is fed into a micro-strain gage (e.g., strain gage 104), as shown in Block A of FIG. 2. A small DC signal is output from the strain gage (e.g., 0 to 15 millivolts), the magnitude of which is dependent upon the force/pressure applied against eyelid “E” by the user. The signal is amplified through an op-amp to produce a DC signal that generally ranges from 0 to 10 Volts, as schematically depicted in Block B. The amplified signal is filtered by a passive low pass filter, as shown in Block C. As shown in Block D, the filtered signal is fed into a breakout box, which delivers such signal to a digital acquisition card (DAQ card) that interfaces the signal to a custom program, e.g., a LabVIEW program.

With further reference to FIG. 2 and as shown in Block E, a function generator feeds an appropriate signal, e.g., a 5 Vrms @ 5 kHz sinusoidal signal, to an LVDT associated with the disclosed tonometer, e.g., LVDT 114. The signal is amplified by an op-amp (shown in Block F) and the amplified signal is generally in the 0 to 10 Volts rms (root mean square) and is rectified by a full wave bridge, as shown in Block G. The rectified signal is filtered to a DC signal by a passive low pass filter (Block H) and the resulting signal is introduced into the breakout box. The signal is thus delivered to the DAQ cared which interfaces to a custom program, e.g., a LabVIEW program, as discussed above.

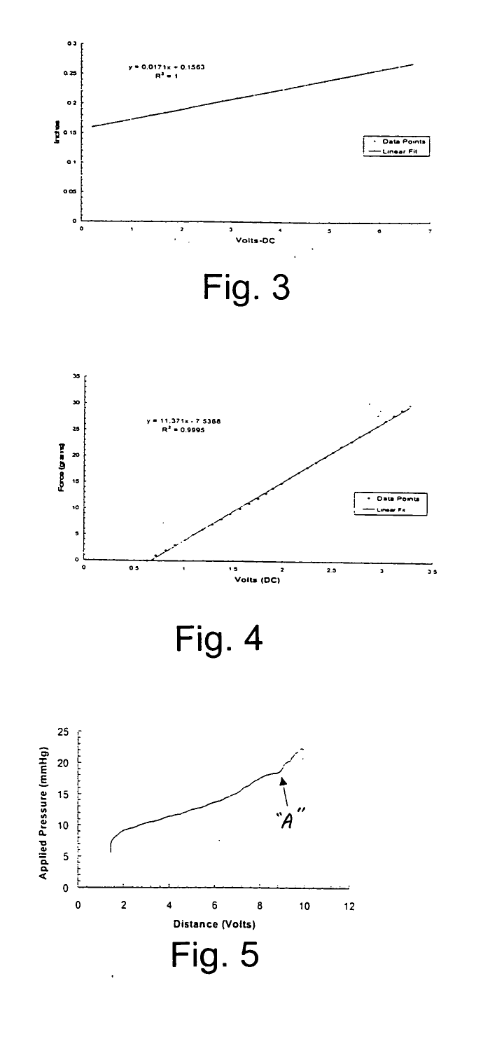

In testing a prototype tonometer fabricated according to the above description, a calibration of both transducers was completed prior to taking any experimental data. For calibration purposes, the LVDT (i.e., LVDT 114) was fixed and a spring was loaded to apply a constant outward force on the armature, i.e., against sensing tip 118. A micrometer was fixed in a head-on position to the armature so that signal output measurements could be recorded with precision down to 0.001 inch. A linear range was established and a LVDT stop 116 was designed and built to ensure that LVDT 114 operates within the desired range. The calibration linearity for the LVDT is shown in FIG. 3.

Also for calibration purposes, the micro strain gage, i.e., strain gage 104, was fixed on a precision digital balance having a resolution of 0.01 grams. A micrometer was fixed above the micro strain gage so that a constant force could be applied with a resulting DC signal. Strain gage stop 106 was designed and built to ensure that the strain gage 104 operates within its mechanical limits. The calibration linearity for the micro strain gage is shown in FIG. 4.

Subsequent to calibration, as described herein, experimental data was obtained using the prototype tonometer fabricated according to the present disclosure. Preliminary graphs show good linear characteristics over an initial range of force application using the prototype tonometer. As the pressure at the sensing tip is increased to, at or above the IOP, the cornea is deformed, causing a reduction in force over a given distance, thereby breaking the linearity and causing a trough-like or inflection area on the graph. Thus, with reference to FIG. 5 which shows the relationship between pressure or force and distance, arrow “A” is directed to the foregoing trough-like/inflection area, where the sensing tip moved without an increase in pressure. The area of the trough/inflection of FIG. 5 denotes a change in the force/pressure relationship and/or the IOP for the test subject. For the experimental study reflected in FIG. 5, the trough/inflection and therefore the subject's IOP fell between 18 and 20 mm Hg. To validate this measurement, the subject had his IOP taken with a Goldmann tonometer (the gold standard for clinical IOP determination), and the IOP measurement was 18 mm Hg. Although these test results showed excellent correlation, some graphs generated from use of the prototype tonometer of the present disclosure with the noted subject showed lower troughs/inflections than the trough/inflection depicted in FIG. 5. It is noted that other techniques or methods for identifying, detecting, observing and/or quantifying the change in the force/distance relationship may also be utilized as will be readily apparent to persons skilled in the art. For example, a change in the force/distance relationship can be visually, audibly, and/or mathematically quantified/represented.

It is contemplated that a correction factor may be developed to better correlate the inflection point with the IOP of a subject. Additional data may facilitate an appropriate modification to the Imbert-Fick law, which is based on the equation W=P*A (where W=external force, P=pressure and A=area). Correction factors are generally implemented to the Imbert-Fick equation in to apply the law to the eye because the eye does not fit all parameters of the law.

Thus, the disclosed tonometer advantageously employs a force gage and a linear potentiometer to identify an inflection point or trough area when a sensing tip is pressed against an eyelid. The disclosed tonometer advantageously time synchronizes the signals received from the force gage and the LVDT with the aid of a processor, thereby permitting the effective identification of the point at which the cornea deforms without actually viewing such deformation. Use of the disclosed tonometer does not require the use of anesthesia, does not cause discomfort, is non-invasive and is easy to use (e.g., a low level of skill is required for use thereof).

Although the advantageous tonometer of the present invention has been described with reference to exemplary embodiments thereof, the present disclosure is not limited to such exemplary embodiments. Rather, changes, modifications and/or enhancements may be made to the disclosed tonometer(s) without departing from the spirit or scope of the present disclosure, as will be readily apparent to persons skilled in the art. For example, it is contemplated that the components of the disclosed tonometer may be combined into a light-weight handheld device. This may be accomplished by combining a micro-electromechanical system (MEMS) type strain gage, MEMS type LVDT, an integrated circuit/chip that operates to handle signal conditioning and data processing, an LED type output screen, and an on-board battery power supply. In addition, it is contemplated that the sensing tip might be fabricated from a metal to provide enhanced resilience to disinfectants and cleansers. Additional changes, modifications and/or enhancements to the disclosed tonometers will be apparent to persons skilled in the art from the description provided herein, and such changes, modifications and/or enhancements are encompassed within the scope hereof

1. A tonometer for use in measuring intraocular pressure in a non-invasive manner, comprising:
(a) a frame;
(b) a strain gage mounted with respect to said frame for measuring a force;
(c) a linear variable displacement transducer mounted with respect to said frame, said linear variable displacement transducer communicating with an axially movable sensing tip for measuring a distance; and
(d) a processing unit in communication with said strain gage and said linear variable displacement transducer, said processing unit being programmed to (i) time-synchronize signals received from said strain gage and said linear variable displacement transducer, and (ii) identify a change in the relationship between time-synchronized measurements of said force and said distance;
wherein said change in the relationship between time-synchronized measurements of said force and said distance correlates with the intraocular pressure of a patient.

2. A tonometer according to claim 1, further comprising a strain gage stop mounted with respect to said frame and positioned adjacent said strain gage.

3. A tonometer according to claim 1, further comprising a linear variable displacement transducer stop mounted with respect to said frame and positioned adjacent said linear variable displacement transducer.

4. A tonometer according to claim 1, wherein said processing unit communicates with said strain gage and said linear variable displacement transducer by way of data communication wires.

5. A tonometer according to claim 1, wherein said frame is substantially L-shaped.

6. A tonometer according to claim 1, wherein said processing unit provides signal amplification.

7. A tonometer according to claim 1, wherein said processing unit provides low pass signal filtering.

8. A tonometer according to claim 1, wherein said processing unit provides signal rectification.

9. A tonometer according to claim 1, wherein said processing unit includes a digital acquisition card that feeds signals to software suitable to time-synchronize the signals received from the strain gage and the linear variable displacement transducer and to identify an inflection or change in the relationship between the time-synchronized force and distance measurements.

10. A tonometer according to claim 9, wherein said software includes spreadsheet functionality.

11. A tonometer according to claim 1, wherein said sensing tip includes a substantially flat circular end for contacting an eyelid of a patient.

12. A tonometer according to claim 1, wherein said strain gage is a micro-electromechanical system.

13. A tonometer according to claim 1, wherein said linear variable displacement transducer is a micro-electromechanical system.

14. A tonometer according to claim 1, wherein said processing unit is embodied in an integrated circuit that is mounted with respect to said frame.

15. A tonometer according to claim 1, further comprising an output screen that includes LED emitters for displaying the intraocular pressure of a patient.
